(19) United States
(12) Patent Application Publication
(10) Pub. No.: US 2004/0242983 A1
(43) Pub. Date: Dec. 2, 2004

(54) Method and device for evaluating measured periodic or quasi-periodic signals produced by medical sensor systems

(76) Inventors: Ralf Bousseljot, Konigs Wusterhausen (DE); Dieter Kreiseler, Berlin (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/490,060

(22) PCT Filed: Sep. 17, 2002
(86) PCT No.: PCT/DE02/03468

(30) Foreign Application Priority Data
Sep. 20, 2001 (DE) 101 46 263.8

(51) Int. Cl. A61B 5/05

(52) U.S. Cl. 600/350

(57) ABSTRACT

The invention concerns a method for evaluating measured periodic or quasi-periodic signals, produced by medical sensor systems, by digitizing the signals and comparing measured signal segments with signal segments stored in a databank. A medical result has been stored and with the stored signal segments and on the basis of the frequency of medical results belonging to the signal segments showing greatest similarity with the measured signal segment, it is possible to determine at least by deduction a medical result corresponding to the measured signal segment. According to the present invention, weighting values corresponding to the probability that the given medical results are those expected are stored with the signal segment data, and a weighting value for each of the medical results concerned is formed on the basis of the weighting values, belonging to the stored signal segments identified as being similar to a measured signal segment, of each of the medical results.

The invention relates to a method for evaluating measured periodic or quasi-periodic signals from medical sensor systems by digitizing the signals and comparing signal portions of the measured signals with signal portions which are stored in a database, the stored signal portions having a medical finding stored with them, and the frequency of the findings associated with the signal portions which are most similar to the measured signal portion being used to draw a conclusion about at least one finding which is associated with the measured signal portion.

The invention also relates to an apparatus for evaluating measured periodic or quasi-periodic signals for medical sensor systems having an analog/digital conversion device for at least signal portions of the measured signals, a database which stores corresponding signal portions together with medical findings which have been established, a comparison device for comparing a measured signal portion with the corresponding stored signal portions in the database and identifying stored signal portions as being similar to the measured signal portion, and having an evaluation device for displaying the frequency of the findings for the signal portions identified as being similar. The inventors have therefore set up a database which has been used to store findings within prescribed findings groups with weighting factors corresponding to the medical diagnosis in the case in question. For an ECG database, a maximum of five findings with the weighting factors 1% to 100% have been permitted in this case. The findings ascertained by comparing a measured 12-channel ECG with the corresponding stored ECGs are thus likewise obtained with weighting factors which characterize the probability of the respective finding being present (R. Bousseljot, D. Kreiseler in “Herzschrittmachertherapie und Elektrophysiologie” [Cardiac pacemaker therapy and electrophysiology], Vol. 11 (2000) 4, pp. 197-206).

The present invention is based on the problem of further improving the automated evaluation using the known method.

To solve this problem, the invention characterizes a method of the type mentioned at the outset in that signals obtained for a plurality of examinations on the same patient which have been carried out at intervals of time are compared with the same stock of stored signal portions and are evaluated, and in that a time profile for the examinations carried out is produced for the ascertained findings.

The present invention allows, for the first time, an objectified comparison between examinations on the same patient which have been carried out at intervals of time. This allows a findings profile for the plurality of examinations, that is to say over a plurality of months or years, for example, to be indicated and possibly displayed. A fundamental aspect of this, in line with the invention, is that the evaluations for ascertaining the findings are respectively carried out in comparison with the same stock of stored signals or signal portions, preferably at the same time. In this regard, it is expedient for a patient's measured signals or signal portions—preferably preprocessed for the automatic evaluation—which have arisen from a plurality of examinations carried out at intervals of time to be stored in relation to this patient, with storage preferably being carried out on a storage card of chip card type associated with the patient or in a storage area associated with the patient in a central store. Expediently, more recent examinations for this patient are added to the storage medium as appropriate, with data for the patient and ascertained findings being stored in addition to the signal portions.

To ascertain the findings, findings groups are expediently formed which may contain primary findings, secondary findings or combinations thereof.

It is particularly expedient to implement the present invention using a database which is used to store, with the data for signal portions, weighting values corresponding to the probability of findings being present, and if the findings' weighting values associated with a measured signal portion from stored signal portions identified as being similar are used to form a weighting value for the respective finding. The weighting values of signal conclusions identified as being similar can be taken into account by summing the entries for the finding in question respectively multiplied by a weighting value of between 0 and 1. It is also possible to form an average weighting value for each finding, with no entry for this finding corresponding to the weighting value 0.

The representation as a bar graph or line diagram, possibly even as a pie chart, allows very clear display of the time profile for individual findings over the examinations carried out at intervals of time, by displaying the corresponding values for the respective finding for the various examinations next to one another.

Such representation may be expedient, in particular, in order to carry out an efficacy control in a patient for administered medicaments.

The invention also solves the problem cited by characterizing an apparatus of the type mentioned at the outset in that an access device is provided on a storage medium which stores signals obtained for a plurality of examinations carried out on the same patient at intervals of time, in that the comparison device compares signals obtained for a plurality of examinations carried out on the same patient at intervals of time with the same stock of stored signal portions, and in that the evaluation device is used to produce a time profile for the ascertained findings over the examinations carried out.

The invention will be explained in more detail below with reference to the appended figures of the drawing, which have been produced for an example patient and in which:

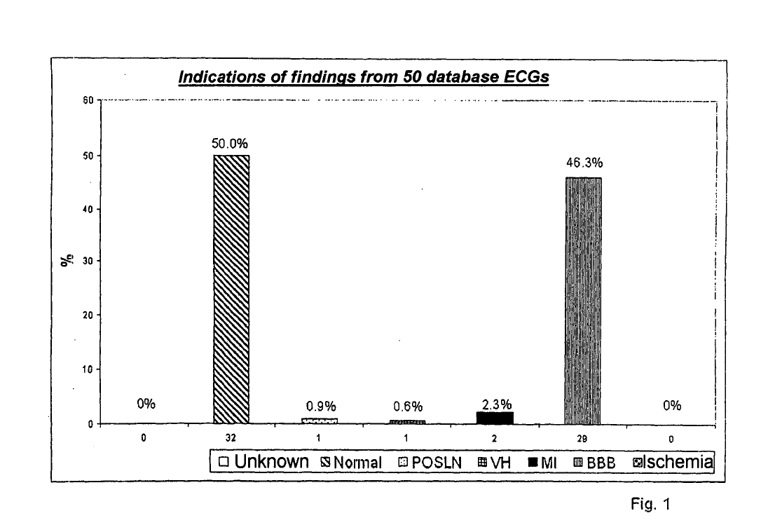

FIG. 1 shows a findings representation for an ECG,

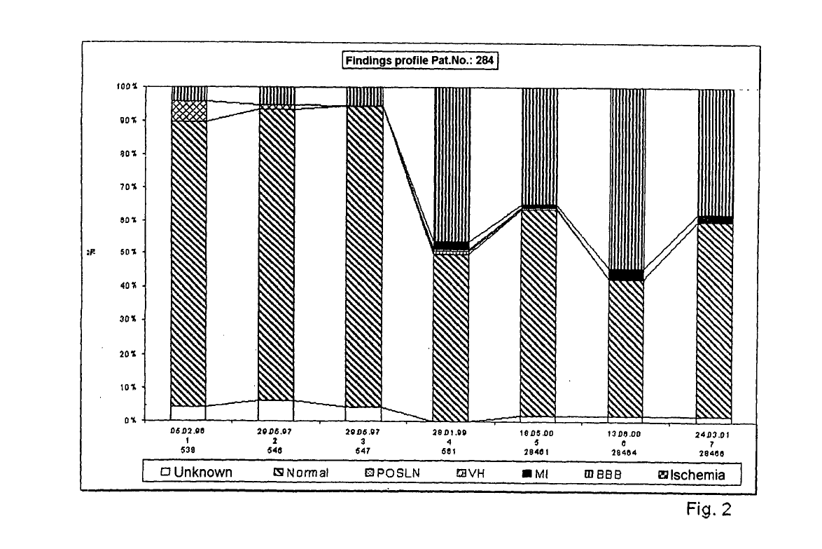

FIG. 2 shows a representation of a findings profile for findings over several years,

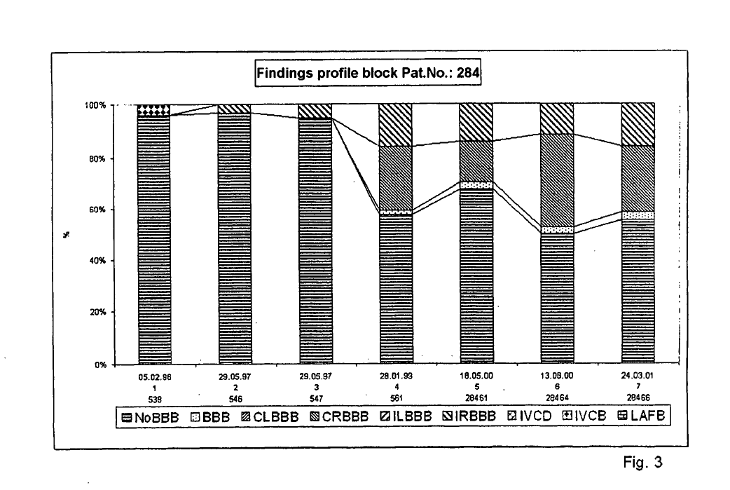

FIG. 3 shows a representation, associated with FIG. 2, of a findings profile for specified sub-findings for a finding,

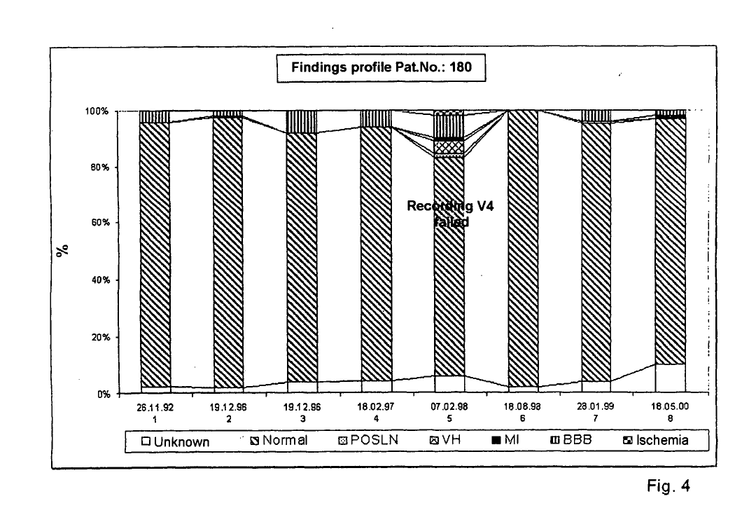

FIG. 4 shows a findings profile for a healthy comparative person,

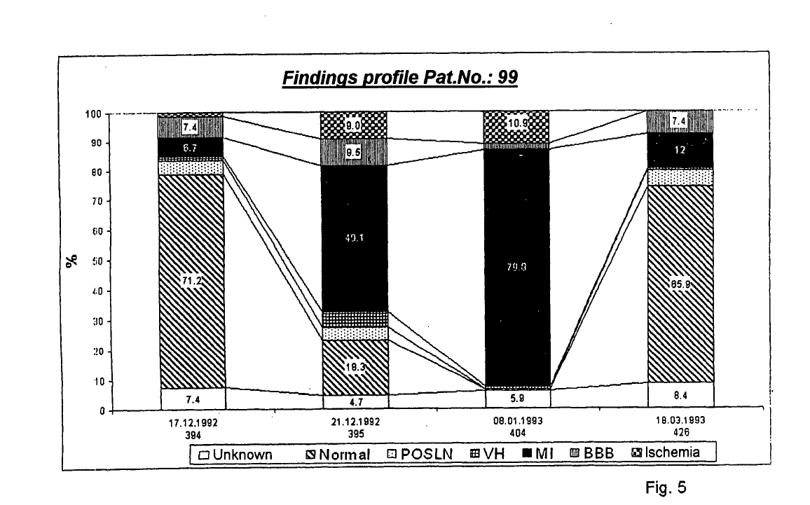

FIG. 5 shows a findings profile for a patient with anterior myocardial infarction,

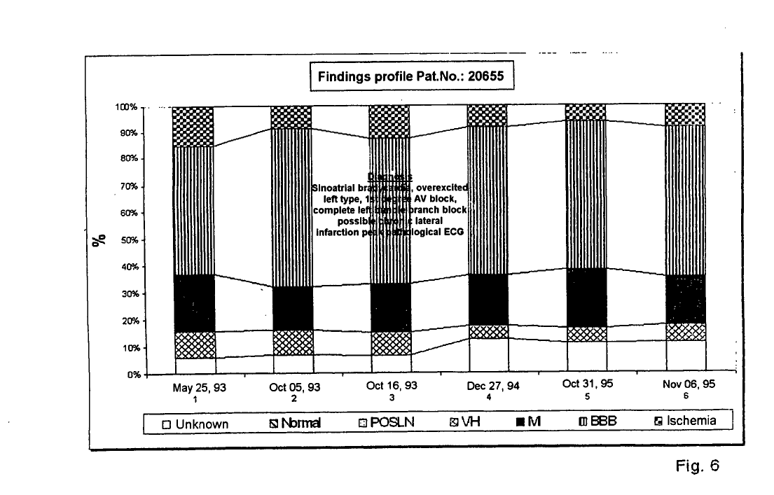

FIG. 6 shows a findings profile for a patient with chronic lateral myocardial infarction,

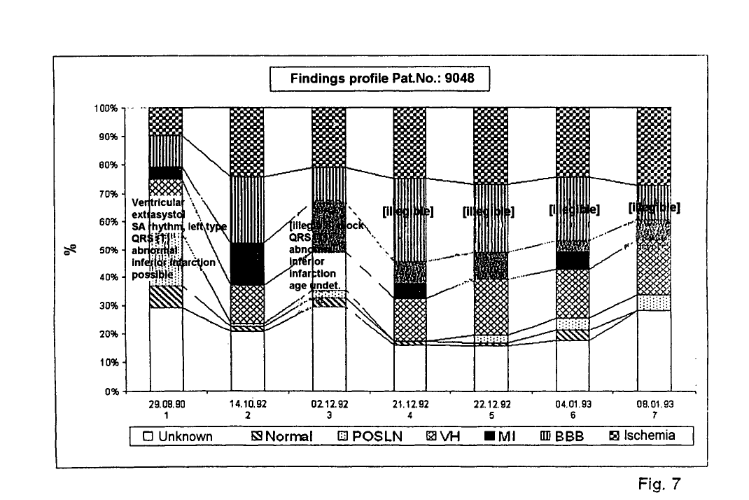

FIG. 7 shows a findings profile for the efficacy control for a medicamentary treatment.

The basis of the inventive method is a signal pattern database, for example an ECG signal pattern database, storing findings for the stored signal patterns with an indication of a weighting value for the findings.

In an ECG database, the following can be regarded as findings:

| | |
|---|---|
| Unkn. | unknown finding |
| NORM | normal finding |
| POSNL. | possibly normal finding |
| VH | ventricular hypertrophy |
| MI | myocardial infarction |
| BBB | bundle branch block |
| ISC | ischemia |

For each stored signal pattern, which in the case of an electrocardiogram ECG comprises numerous signal patterns for the usual recordings, the findings will use the weighting values, for example in the form of weighting factors, for assessment, with exclusion of this finding being given the weighting factor 0% and an absolutely safe finding being given the weighting factor 1 (100%). Intermediate values may exist for “cannot be excluded, but rather unlikely” 15%, “possible, because certain symptoms are present” 35% etc.

FIG. 1 shows a representation of findings for a patient taking into account 50 database signal patterns which have been found to be similar. The ascertained weighting values shown as bars in FIG. 1 are obtained from the number of entries for the corresponding finding multiplied by the respective weighting factor, and formation of a corresponding sum.

Of the 50 database signal patterns identified as being similar, 32 contained the finding “normal” and 29 contained the indication of a block (BBB). Taking into account the weighting factors which are also stored, a weighting value for “normal” of 50.0% is obtained and a weighting value for “BBB” of 46.3% is obtained. One entry was found for “POSNL.”, one entry was found for “VH” and two entries were found for “MI”. Taking into account the stored weighting values, a weighting value of 0.9% is obtained for “POSNL.”, a weighting value of 0.6% is obtained for “VH”, and a weighting value of 2.3% is obtained for “MI”. These three values are below a relevant cardiological finding, which means that the representation of findings permits a conclusion to the effect that the patient has a block, that is to say a defect in the heart's conduction system, but that further cardiological findings are not available. The inventive method affords considerable advantages when a plurality of overlapping findings arise. In this case, a probability, a weighting value, can be indicated quantitatively for each of the findings in relation to the other findings. This statement was not possible with previous ECG evaluation methods.

Previous evaluation methods also had only limited ability to make statements about whether a finding has changed in comparison with previous ECG recordings. It was possible to assess the change in particular signal parameters of the ECG, e.g. the reduction in the R wave amplitude over the increase in the QRS period.

The inventive method permits a clear representation of a findings profile.

FIG. 2 shows the findings profile for the patient from FIG. 1 over seven examinations in the course of five years.

FIG. 2 reveals that examinations 1 to 3 from 1996/1997 produced an essentially normal ECG. For examination 4 in 1999, a sudden block-typical change is obtained, whose intensity varies in the subsequent ECG recordings 5 to 7. Examinations 4 to 7 produce a block finding whose trend is more amplified.

FIG. 3 shows the findings profile for details relating to sub-findings and reveals that, in particular, the findings component CRBBB (complete right bundle branch block) is increasing. The sub-findings groups are:

| | |
|---|---|
| CLBBB | complete left bundle branch block |
| CRBBB | complete right bundle branch block |
| ILBBB | incomplete left bundle branch block |
| IRBBB | incomplete right bundle branch block |
| IVCD | unspecific intraventricular conduction |
| | defect |
| IVCB | unspecific intraventricular conduction block |
| LAFB | left anterior fascicular block |

The findings profile in FIGS. 2 and 3 reveals that the block has developed, that is to say represents no prior illness. The addition of further ECG measurements allows the further evolution of the findings profile to be tracked quantitatively. This was not possible with ECG evaluation methods previously.

The availability of the ECG signals recorded at an earlier time can be provided by virtue of the ECG signals being available on a central data server, e.g. belonging to the resident doctor or the cardiologist. Alternatively, the ECG data may be held on a chip card belonging to the patient, which affords advantages in relation to data integrity.

The inventive method also works as a client-server solution over the Internet and can therefore be applied virtually from any location. Only the ECG measured data need to be transmitted, which, as data without any graphics components, comprise only a small data volume.

The invention affords the option of not only establishing changes in the ECG but also of simultaneously obtaining a statement about the findings which are affected by the changes.

A prerequisite of the inventive method is naturally that the ECG measurement be taken without any errors. In the case of the exemplary embodiment shown in FIG. 4, the findings profile is shown for a healthy comparative person, with a recording electrode (V4) having failed during measurement 5 on 02.07.1998. This failure naturally affects the calculated finding for this measurement.

FIG. 5 shows, by way of example, the findings profile for a patient with an anterior myocardial infarction over the period of three months. Following the first signs of infarction (measurement on 12.17.1992), the extent of the infarction (measurements on 12.21.1992 and, in particular, 01.08.1993) is clear. The signs of infarction then start to subside and the finding begins to normalize (measurement on 03.18.1993).

FIG. 6 shows the findings profile for a chronic lateral myocardial infarction over a period of 2.5 years. The state does not change significantly.

FIG. 6 indicates that the inventive method allows an efficacy control for a medicamentary treatment using the ECG. Findings 1 to 3 in FIG. 7 indicate that an abnormal inferior infarction has occurred. Following treatment with a plurality of medicaments, the findings profile based on measurements 4 to 7 is obtained, allowing the doctor to obtain evaluatable indications.

1. A method for evaluating periodic or quasi-periodic signals, measured during examination of a patient, from a medical sensor system by digitizing the signals and comparing signal portions of the measured signals with signal portions which are stored in a database, the stored signal portions having a medical finding stored with them, and the frequency of the findings associated with the signal portions which are most similar to the measured signal portion being used to draw a conclusion about at least one finding associated with the measured signal portion, characterized in that signals obtained for a plurality of examinations on the same patient which have been carried out at intervals of time are compared with the same stock of stored signal portions and are evaluated, and in that a time profile for the examinations carried out is produced for the ascertained findings.

2. The method as claimed in claim 1, characterized in that the findings for the stored signal portions and the ascertained findings are determined within a same set of findings groups.

3. The method as claimed in claim 1, characterized in that weighting values corresponding to the probability of findings being present are stored with the data for signal portions, and in that the findings' weighting values associated with a measured signal portion from stored signal portions identified as being similar are used to form a weighting value for the respective finding.

4. The method as claimed in claim 1, characterized in that the signals obtained for the examinations carried out at intervals of time are stored and compared with the same stored signal portions at the same time.

5. The method as claimed in claim 4, characterized in that the signals measured for a patient during the individual examinations are stored with further patient-related data and findings in association with the patient.

6. The method as claimed in claim 4, characterized in that the storage medium used is a storage card.

7. The method as claimed in claim 1, characterized in that the temporal relationship between the ascertained findings is portrayed comparatively for the patient's signals measured at intervals of time.

8. The method as claimed in claim 7, characterized in that the temporal relationship between the ascertained findings is displayed graphically.

9. The method as claimed in claim 9, characterized in that the graphical display takes the form of bar graphs, line diagrams or pie charts.

10. An apparatus for evaluating measured periodic or quasi-periodic signals for medical sensor systems having an analog/digital conversion device for at least signal portions of the measured signals, a comparison device for comparing a measured signal portion with corresponding signal portions stored in a database, which stores corresponding signal portions together with medical findings which have been established, and identifying stored signal portions as being similar to the measured signal portion, and having an evaluation device for displaying the frequency of the findings for the signal portions identified as being similar, characterized in that an access device is provided on a storage medium which stores signals obtained for a plurality of examinations carried out on the same patient at intervals of time, in that the comparison device compares signals obtained for a plurality of examinations carried out on the same patient at intervals of time with the same stock of stored signal portions, and in that the evaluation device is used to produce a time profile for the ascertained findings over the examinations carried out.

11. The apparatus as claimed in claim 10, characterized by a writing device for writing signals obtained for the patient from a new measurement to the storage medium.

12. The apparatus as claimed in claim 10, characterized in that the storage device is a transportable storage device associated with the patient.

13. The apparatus as claimed in claim 12, characterized in that the transportable storage device is a storage card.

14. The apparatus as claimed in claim 10, characterized in that the storage medium is part of a storage unit which is physically remote from the patient's location and can have information read from and/or written to it using telecommunication devices.

15. The apparatus as claimed in claim 10, characterized in that the evaluation device is set up to form a weighting value from weighting values stored in the database for findings.

16. The apparatus as claimed in claim 10, characterized by a display for graphically displaying the temporal relationship between the findings from the examinations and also, if appropriate, further information associated with the measurements.
